(19) United States
(12) Patent Application Publication
(10) Pub. No.: US 2024/0423757 A1
(43) Pub. Date: Dec. 26, 2024

(54) APPARATUSES AND METHODS ASSISTING IN DENTAL THERAPIES

(71) Applicant: Align Technology, Inc., San Jose, CA (US)

(72) Inventor: Christopher Eric CRAMER, Durham, NC (US)

(21) Appl. No.: 18/807,905

(22) Filed: Aug. 16, 2024

Related U.S. Application Data
(62) Division of application No. 15/937,569, filed on Mar. 27, 2018, now Pat. No. 12,090,020.
(60) Provisional application No. 62/477,389, filed on Mar. 27, 2017.

(51) Int. Cl. A61C 7/00; A61C 7/08; A61C 19/04; B33Y 50/00; B33Y 80/00; G16H 10/60; G16H 20/30; G16H 20/40; G16H 50/20; G16H 50/50

(52) U.S. Cl. CPC A61C 7/002; A61C 19/04; G16H 10/60; G16H 20/30; G16H 20/40; G16H 50/20; A61C 2007/004; A61C 7/08; B33Y 50/00; B33Y 80/00; G16H 50/50

(57) ABSTRACT

Provided herein are methods and apparatuses for recommending tooth leveling (e.g., one or more of anterior leveling, posterior leveling, and arch-shape recommendations) for orthodontic devices and methods for patients. Factors involved in the leveling of teeth can include symmetry, doctor preferences, preferences regarding gender, the country in which the patient is being treated, and other issues related to the aesthetics of the mouth and arch shape. The device can comprise an aligner configured to fit over a patient's teeth. Methods of designing and manufacturing aligners based on leveling recommendations are also provided.

### CROSS REFERENCE TO RELATED APPLICATIONS

This application is a continuation of U.S. patent application Ser. No. 15/937,569, filed Mar. 27, 2018, now U.S. Patent Application Publication No. 2018/0280118, which claims priority to U.S. Provisional Patent Application No. 62/477,389, filed Mar. 27, 2017, each of which is herein incorporated by reference in its entirety.

### INCORPORATION BY REFERENCE

All publications and patent applications mentioned in this specification are incorporated herein by reference in their entirety to the same extent as if each individual publication or patent application was specifically and individually indicated to be incorporated by reference.

### BACKGROUND

Orthodontic procedures typically involve repositioning a patient's teeth to a desired arrangement in order to correct malocclusions and/or improve aesthetics. To achieve these objectives, orthodontic appliances such as braces, shell aligners, and the like can be applied to the patient's teeth by an orthodontic practitioner and/or by the patients themselves. The appliance can be configured to exert force on one or more teeth in order to effect desired tooth movements according to a treatment plan.

Orthodontic aligners may include devices that are removable and/or replaceable over the teeth. Orthodontic aligners may be provided as part of an orthodontic treatment plan. In some orthodontic treatment plans involving removable and/or replaceable aligners, a patent may be provided plurality of orthodontic aligners over the course of treatment to make incremental position adjustments to the patient's teeth. An orthodontic aligner may have a polymeric trough with an inner cavity shaped to receive and resiliently reposition teeth from one tooth arrangement to a successive tooth arrangement. Orthodontic aligners may include “active” regions that impose repositioning forces on teeth and “passive” regions that retain teeth in their current state.

Many orthodontic treatment plans, including at least some of those that involve removable and/or replaceable appliances that provide incremental forces on teeth over time, include a determination of tooth leveling. Tooth leveling may involve the extent to which a person's teeth are intruded or extruded relative to their gums. Under many orthodontic treatment plans, tooth leveling may be determined independently of other aspects of determination of final position(s) of a patient's teeth. Some factors influencing tooth leveling are aesthetics (which, in many orthodontic treatment plans may primarily affect anterior tooth positioning) and clinical positioning (which may primarily affect posterior teeth). A tooth leveling determination may also depend on what a given doctor thinks is appropriate for a patient as well as characteristics of the patient (patient gender, patient heritage, patent location, etc.). Existing systems do not effectively provide a treatment professional with recommendations of tooth levels appropriate for a patient, much less offer treatment professionals the ability to design orthodontic appliances to accommodate and/or visualize the effects of tooth level recommendations.

### SUMMARY

Systems, methods, and/or computer-readable media described herein provide technical solutions to the highly technical problems of machine visualization of tooth leveling recommendations. “Tooth leveling,” as used herein, may refer to a parameter for identifying the extent a tooth is intruded and/or extruded from a patient's gums. Tooth leveling may be achieved by a variety of ways, including application of incremental forces to a tooth over a specified period of time. Tooth leveling may generally refer to adjusting/modifying the position (e.g., the z-component of position) of a tooth or teeth. Tooth leveling may include anterior leveling (e.g., leveling of the anterior teeth, e.g., incisors and canines), posterior leveling (e.g., leveling of posterior teeth, e.g., premolars and molars, and/or arch shape. The methods and apparatuses described herein may include one or more of: anterior leveling, posterior leveling or arch shape adjustment. Thus, “tooth leveling” may refer to one or more of these (in combination). In some variations, it may be beneficial to separately perform methods for anterior leveling and posterior leveling of teeth; as mentioned, a recommendation for tooth leveling may include both anterior tooth leveling and posterior tooth leveling (and/or arch shape) or it may include just one or two of these.

Systems, methods, and/or computer-readable media explored herein train automated agents to learn latent leveling factors to associate one or more specific leveling recommendations with specific patient types. An “automated agent,” as used herein, may refer to one or more computer-program instructions, which when accessed by a computer processor, execute one or more computer-implemented methods without human intervention.

A “latent leveling factor,” as used herein, may refer to a factor, unknown at the time of training, that forms the basis of a significant association between specific treatment professional(s) and specific historical leveling recommendations those treatment professional(s) have provided to patients. A “latent leveling factor,” in some implementations, may also refer to a factor, unknown at the time of training that forms the basis of a significant association between specific patient type and specific historical leveling recommendations for that patient type. A “leveling recommendation,” as used herein, may refer to a recommended tooth leveling provided to a patient as part of a prescription. A “historical leveling recommendation,” as used herein, may refer to a leveling recommendation that was implemented on a specific patient and/or group of patients. A historical leveling recommendation may be contrasted with an “estimated” or “derived” leveling recommendation that is to be prescribed to a patient. A “patient type,” as used herein, may refer to a characteristic (gender, country of origin, age, jaw parameter range, face parameter range, arch parameter range, malocclusion characteristic, etc.) common to two or more patients. A “treatment professional,” used interchangeably herein with “doctor,” “orthodontist,” “dentist,” etc., may refer to any individual who implements a treatment plan, such as an orthodontic or restorative treatment plan.

In some implementations, a first set of historical leveling parameters are gathered. One or more of the first set of historical leveling parameters may be associated with a first set of treatment professionals. In some implementations, the first set of historical leveling parameters may include leveling parameters that the first set of treatment professionals have prescribed and/or implemented on a first set of patients. In various implementations, a second set of historical leveling parameters may be gathered. One or more of the second set of historical leveling parameters may be associated with a first set of patient types of patients who have undergone a treatment plan. The second set of historical leveling parameters may include leveling parameters implemented on the patent type of the first set of patients in the past.

A set of latent leveling factors may be derived from the first dataset and the second dataset. One or more of the latent leveling factors may provide a latent statistical basis (e.g., may be associated in a statistically significant way) to associate the first set of historical leveling parameters with the first set of doctors in the first dataset. One or more of the latent leveling factors may further provide a latent statistical basis to associate the second set of historical leveling parameters with the patient types of the first set of patients in the second dataset. Operations may be taken to regularize the set of latent leveling factors by, e.g., whether one or more of the latent leveling factors exceeds a complexity threshold, and removing any latent leveling factors exceeding the complexity threshold. Operations may also be taken to identify whether or not the one or more of the latent leveling factors correspond to bias by seeing if the latent leveling factors deviate from a known correlation between the first set of historic leveling parameters and the second set of historic leveling parameters.

The latent leveling factors may be used to derive a leveling recommendation for one or more combinations of doctors from the set of first doctors and one or more patient types from the one or more first set of patients. In some implementations, the latent leveling factors allow the combinations of doctor/patient type pairs to be associated with specific leveling recommendations. Advantageously, these latent leveling factors may be learned through training the automated agents described herein. One or more leveling recommendations may be stored in a leveling recommendation database as described further herein.

In various implementations, a leveling recommendation is identified for a specific patient as part of final dental position calculations and/or visualizations for that patient. The leveling recommendation may include recommended tooth levels for the outcome of an orthodontic prescription. The leveling recommendation may include specific parameters to identify the extent the patient's teeth will be extruded and/or intruded from their gums after the orthodontic treatment plan. The leveling recommendation may be provided in conjunction with, or independent of, other final dental position parameters, such as tooth location(s), orientation(s), etc.

In various implementations, the patient data may be associated with a doctor identifier of a doctor who is implementing the treatment plan. The doctor identifier may correspond to the specific doctor implementing the treatment plan or may correspond to one or more doctors who have provided prescriptions to other patients similarly situated to the patient.

In some implementations, patient data may be gathered. The patient data may include information about the patient's gender, country of origin, age, jaw parameter range, face parameter range, arch parameter range, malocclusion characteristic, etc. The patient data may be used to associate the patient with a patient type identifier. The patient type identifier may use one or more patient characteristics to group the patient with other patients who have undergone or will undergo orthodontic treatment. As an example, the patient type identifier may group the patient with other patients based on one or more of gender, country of origin, age, jaw parameter range, face parameter range, arch parameter range, malocclusion characteristic, etc.

The doctor identifier and the patient identifier may be used to identify a leveling recommendation for the patient. As noted herein, the leveling recommendation may be derived from one or more latent leveling factors using automated agents to learn those latent leveling factors based on historic leveling parameters used in historic populations of patients by historic populations of treatment professionals. The doctor and/or the patient may be provided with instructions to display the leveling recommendation in a format that is convenient to their orthodontic treatment plan(s). In various implementations, the leveling recommendations may be incorporated into one or more tooth positions displayed in treatment visualization software for the doctor and/or patient. As noted herein, the tooth positions may include and/or be compatible with tooth parameters, implicit ratings, vector ratings, multiple ratings for a patient, and/or a larger leveling model configured to be displayed on a computer to the doctor and/or the patient.

In general, described herein are methods and apparatuses (including systems, devices and software, hardware, and/or firmware) for providing recommendations, and in particular, recommendations that are tailored to a particular health care provider, for treating a particular patient. In particular, described herein a apparatuses and methods for providing a particular, specified health care provider with recommendation for orthodontic treatment, including leveling of one or more teeth. As used herein, a health care provider may refer to a physician, nurse, dentist, orthodontist, technician, etc., and may for the sake of simplicity be referred to herein as a “doctor”.

In some embodiments, anterior tooth leveling may generally include creating a model based on known or assumed factors regarding doctors and patients and then capturing the residual error using a factorized matrix. This may provide an accurate prediction for anterior leveling that should be desirable to the specific doctor being provided with the recommendation. Similarly, posterior leveling may generally include analysis of previous cases (for a particular doctor) in order to determine the factors involved in posterior leveling; these factors may then be used to evaluate a proposed leveling, e.g., using either a Naïve Bayes or a Tree-augmented Naïve Bayes model. By optimizing over the scorer, a recommendation can be generated. Finally, arch shape recommendations may recommend an arch shape for a given doctor and type of patient using a combination of elliptic Fourier descriptors and a collaborative filter based on matrix factorization (e.g., matrix factorization in 3 dimensions) in order to predict Elliptic Fourier Descriptor (EFD) components that are doctor and patient specific.

In some embodiments, described herein are methods for providing a leveling recommendation to a doctor for a patient's teeth, the method comprising: inputting into a computing system an initial anterior leveling dataset of the patient's anterior teeth; inputting into the computing system an incisor preference (b_{pref}) received from the doctor regarding a leveling position of lateral incisors; obtaining with the computing system an average anterior leveling (μ) dataset derived from a plurality of patients; determining a final leveling dataset based on the initial anterior leveling dataset, the incisor preference received from the doctor and the average anterior leveling dataset; and displaying, to the doctor, a final leveling recommendation from the final leveling dataset.

These methods may also include determining leveling (e.g., a leveling dataset) including the posterior teeth as well, although in some variations, the posterior teeth may be excluded. Any of these methods may also include generating an orthodontic device from the final leveling dataset. For example, a method of providing a leveling recommendation may also include determining a posterior leveling dataset based on the patient's posterior teeth, and determining the dataset may comprise determining the final leveling data set based on the initial anterior leveling dataset, the incisor preference received from the doctor, the average anterior leveling dataset, and the posterior leveling dataset.

In any of the methods described herein, the final leveling recommendation (e.g., anterior, posterior, arch shape) may be displayed on the computing device, may be displayed as an output (visual, digital, printout), or may be displayed as a model (virtual, 3D fabricated, etc.) of the teeth and/or a dental appliance to be used on the teeth.

Determining the final leveling dataset may be based on at least one latent factor, wherein the at least one latent factor comprises one or more of: a country bias, a gender bias, a patient-type bias, and a doctor/gender bias. In particular, the latent factor may be a doctor/gender bias (e.g., p_{dg}).

The incisor preference (b_{pref}) may be received from the doctor regarding the leveling position of lateral incisors with respect to a leveling position of central incisors. This b_{pref} may be a preference by the doctor that the lateral incisors be raised with respect to the central incisors, and/or a preference by the doctor that the lateral incisors be level with the central incisors, and/or a preference by the doctor that the lateral incisors and central incisors be leveled based gingival margins of the patient.

Determining a final leveling dataset may include or involve applying matrix factorization to matrices containing the initial anterior leveling dataset, the incisor preference (b_{pref}), and the average anterior leveling dataset (μ).

The final leveling dataset may comprise a matrix containing a set of possible leveling recommendations and a score for each leveling recommendation. Any of these methods may include selecting the final leveling recommendation from the final leveling dataset. The selected final leveling recommendation may comprise a highest score from the set of possible leveling recommendations. The final leveling recommendation may be added to the average anterior leveling dataset.

As another example of a method for providing a leveling recommendation, described herein are methods for providing an anterior leveling recommendation to a doctor for a patient's teeth. The method may include: inputting into a computing system an initial anterior leveling dataset of the patient's anterior teeth; inputting into the computing system an incisor preference (b_{pref}) received from the doctor regarding a leveling position of lateral incisors; obtaining with the computing system an average anterior leveling (μ) dataset derived from a plurality of patients; determining a final anterior leveling dataset based on the initial anterior leveling dataset, the incisor preference received from the doctor and the average anterior leveling dataset; and displaying, to the doctor, a final anterior leveling recommendation from the final leveling dataset.

A method for providing an arch shape recommendation to a doctor for a patient's dental arch may include: inputting into a computing system an initial arch shape dataset of the patient's dental arch; grouping the patient into a patient class based on the initial arch shape dataset; obtaining with the computing system an average arch shape dataset for the patient class; determining, with the computing system, a patient bias based on an average residual of arch shape recommendations for the patient class; determining, with the computing system, a doctor bias based on average residual of arch shape recommendations for the patient class made by the doctor; determining a final arch shape dataset based on the initial arch shape dataset, the patient bias, and the doctor bias; and displaying, to the doctor, a final arch shape recommendation from the final arch shape dataset. The methods for providing an arch shape recommendation may be part of a general method for providing a leveling recommendation to a doctor for a patient's teeth.

Determining the final arch shape may comprise applying matrix factorization to matrices containing the initial arch shape dataset, the patient bias, the doctor bias, and the average arch shape dataset. The final arch shape dataset may comprise a matrix containing a set of possible arch shape recommendations and a score for each arch shape recommendation.

Also described herein are methods for providing a leveling recommendation to a doctor for a patient's posterior teeth. For example, a method of providing a leveling recommendation to a doctor for a patient's posterior teeth may include: inputting into a computing system an initial posterior leveling dataset of the patient's posterior teeth; obtaining with the computing system an average posterior leveling dataset from a plurality of patients; determining a final posterior leveling dataset based on the initial posterior leveling dataset, the average posterior leveling dataset, and at least one factor from the initial posterior leveling dataset; and selecting a final posterior leveling recommendation from the final posterior leveling dataset. The methods for providing a leveling recommendation to a doctor for a patient's posterior teeth may be part of a general method for providing a leveling recommendation to a doctor for a patient's teeth. The at least one factor may comprise a leveling difference between corresponding teeth on a lower jaw and an upper jaw of the patient, and/or a leveling difference between corresponding teeth on a left and right side of a jaw of the patient, and/or an absolute leveling of at least one tooth. Determining may comprise using classifier theory to determine a probability distribution representing whether a given posterior leveling recommendation will be accepted by the doctor.

The selected final posterior leveling recommendation may comprise the highest probability from probability distribution. The final anterior leveling recommendation may be added to the average posterior leveling dataset.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A shows an example of a system for estimating leveling recommendations using application of historical leveling parameters associated with doctors and application of historical leveling parameters associated with patient types.

FIG. 1B shows an example of a system for generating leveling recommendations derived from latent leveling factors.

FIG. 1C shows an example of a method for estimating leveling recommendations using application of historical leveling parameters associated with doctors and application of historical leveling parameters associated with patient types.

FIG. 1D shows an example of a method for generating leveling recommendations derived from latent leveling factors.

FIG. 1E illustrates an exemplary flowchart of a method for providing a leveling recommendation to a doctor for a patient's teeth as described herein.

FIG. 2A is a graphical representation of the Naïve Bayes model.

FIG. 2B shows an example of the initial distribution of levelings for tooth 4 and the accepted distribution of levelings.

FIG. 3 is a sample distribution of upper to lower leveling for upper teeth 4 and 13 and their corresponding lower jaw teeth, 29 and 20, respectively.

FIG. 4 shows facial symmetry sample distributions including an initial leveling symmetry distribution between teeth 5 and 12, a corrected symmetry distribution, and the change in symmetry after correction.

FIG. 5 shows a sample distribution over the change in leveling seen from initial to accepted in tooth 5.

FIG. 6 shows a spanning tree that models dependence between variables using mutual information. The node labels indicate tooth number.

FIG. 7 is a simplified block diagram of a data processing system for designing and manufacturing an orthodontic aligner using leveling recommendations derived from latent leveling factors.

FIG. 8 shows an orthodontic aligner manufactured according to the algorithms described herein.

### DETAILED DESCRIPTION

The present disclosure is related to systems, methods, computing device readable media, and devices for creating an orthodontic aligner.

The planning and fabrication of such dental appliances as an example clastic polymeric positioning appliance is described in detail in U.S. Pat. No. 5,975,893, and in published PCT application WO 98/58596 which designates the United States, and which is herein incorporated by reference for all purposes. Systems of dental appliances employing technology described in U.S. Pat. No. 5,975,893, are commercially available from Align Technology, Inc., Santa Clara, Calif., under the tradename, Invisalign System. Align Technology, Inc.

Throughout the body of the Detailed Description, the use of the terms “orthodontic aligner”, “aligner”, or “dental aligner” is synonymous with the use of the terms “appliance” and “dental appliance” in terms of dental applications. For purposes of clarity, embodiments are hereinafter described within the context of the use and application of appliances, and more specifically “dental appliances.”

Factors involved in the leveling of teeth include symmetry, doctor preferences, preferences regarding gender, the country in which the patient is being treated, and other issues related to the aesthetics of the mouth and arch shape.

In general, described herein are methods for providing a leveling recommendation to a doctor for a patient's teeth. These leveling methods may be used for providing one or more of: anterior leveling recommendation, posterior leveling recommendations, and arch shape recommendations.

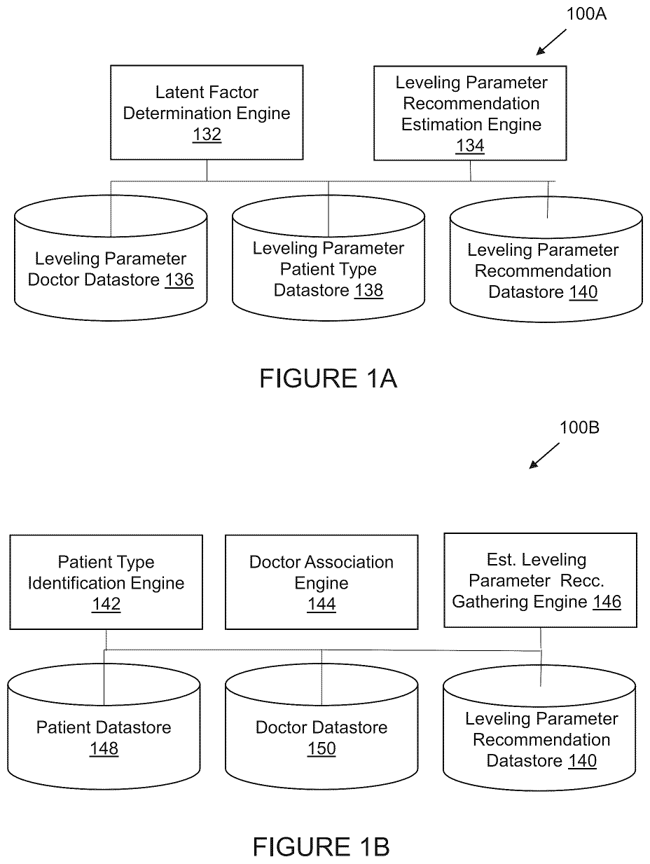

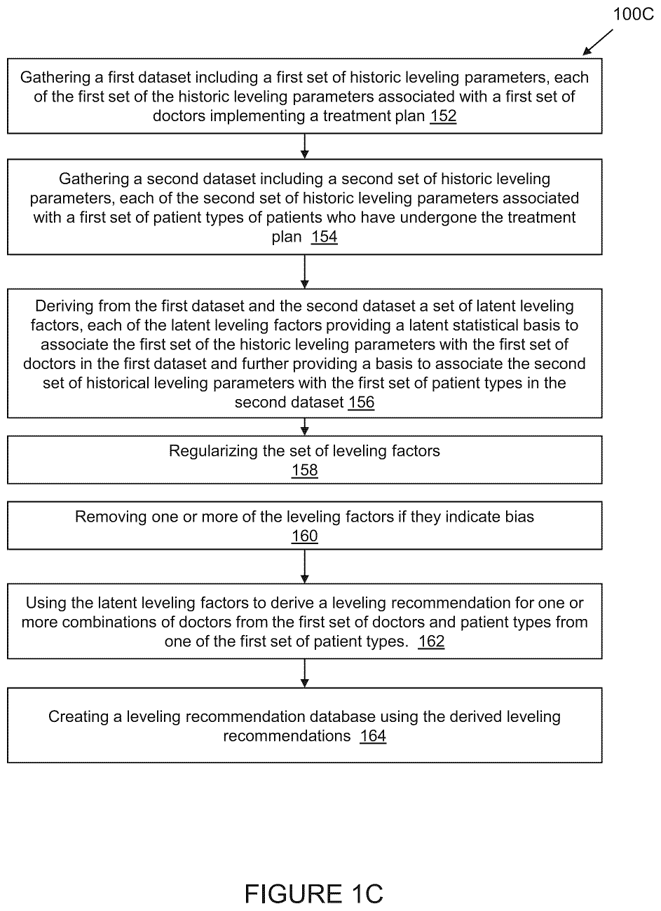

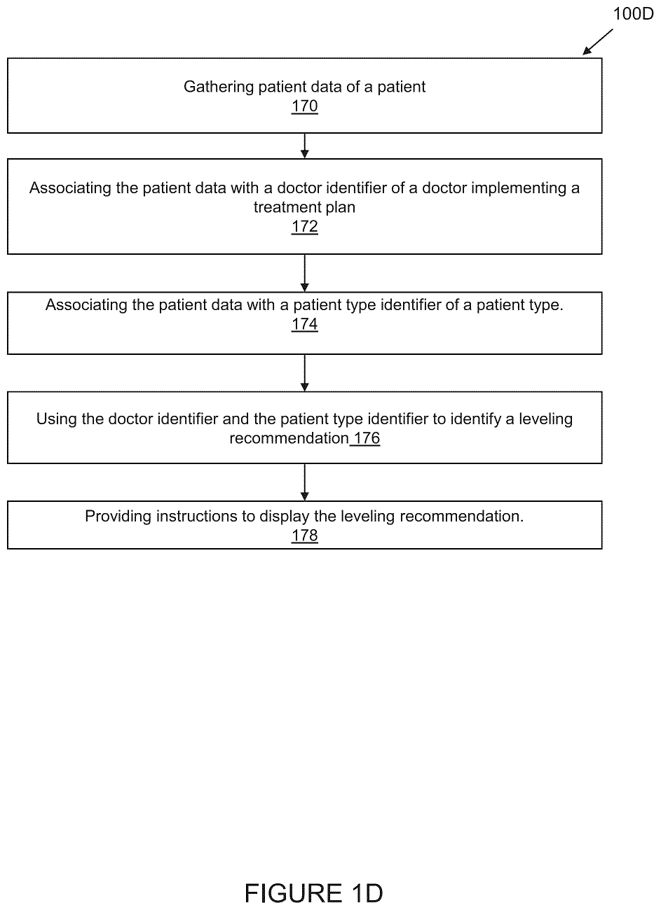

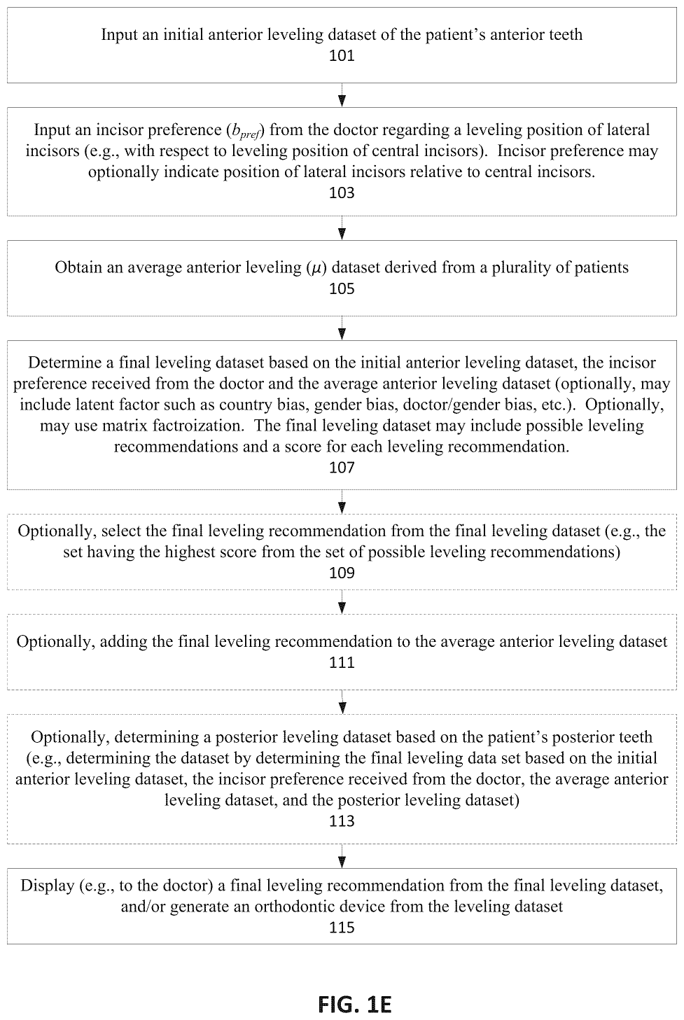

FIG. 1A shows an example of a system 100A for estimating leveling recommendations using application of historical leveling parameters associated with doctors and application of historical leveling parameters associated with patient types. The system 100A may include engines and/or datastores. A computer system can be implemented as an engine, as part of an engine or through multiple engines. As used in this paper, an engine includes one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors or a first engine and a second engine can share one or more processors with one another or other engines. Depending upon implementation-specific or other considerations, an engine can be centralized or its functionality distributed. An engine can include hardware, firmware, or software embodied in a computer-readable medium for execution by the processor. The processor transforms data into new data using implemented data structures and methods, such as is described with reference to the FIGS. in this paper.

The engines described in this paper, or the engines through which the systems and devices described in this paper can be implemented, can be cloud-based engines. As used in this paper, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

As used in this paper, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described in this paper.

Datastores can include data structures. As used in this paper, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described in this paper, can be cloud-based datastores. A cloud-based datastore is a datastore that is compatible with cloud-based computing systems and engines.

In the example of FIG. 1A, the system 100A includes a latent factor determination engine 132, a leveling parameter recommendation engine 134, a leveling parameter doctor datastore 136, a leveling parameter patient type datastore 138, and a leveling parameter recommendation datastore 140. One or more of the modules of the system 100A may be coupled to one another or to modules not explicitly shown.

The latent factor determination engine 132 may be configured to estimate latent leveling factors using historical leveling parameters indexed by doctor and historical leveling parameters indexed by patient type. In some implementations, the latent factor determination engine 132 gathers first set of historic leveling parameters, each of the first set of the historic leveling parameters associated with a first set of doctors implementing a treatment plan. The latent factor determination engine 132 may further gather a second set of historic leveling parameters, where each of the second set of historic leveling parameters is associated with a first set of patient types of patients who have undergone the treatment plan.

The latent factor determination engine 132 may further be configured to derive from the first set of historical leveling data and the second set of historical leveling data a set of latent leveling factors that were used as the basis of prescribing the first and second set of historical data. One or more of the latent leveling factors may provide a latent statistical basis to associate the first set of the historic leveling parameters with the first set of doctors in the first dataset. One or more of the latent leveling factors may provide a basis to associate the second set of historical leveling parameters with the first set of patient types in the second dataset. The latent factor determination engine 132 may use automated agents to identify these latent leveling factors using techniques such as sparse-matrix factorization of the first dataset against the second dataset or vice versa.

The latent factor determination engine 132 may be configured to regularize the set of latent leveling factors. For instance, the latent factor determination engine 132 may be configured to determine whether one or more of the latent leveling factors exceeds a complexity threshold, and removing any latent leveling factors exceeding the complexity threshold. In some implementations, the latent factor determination engine 132 may be configured to identify whether one or more of the latent leveling factors correspond to bias by seeing if the latent leveling factors deviate from a known correlation between the first set of historic leveling parameters and the second set of historic leveling parameters. Examples of regularization and bias determination techniques are described in great detail herein.

The leveling parameter recommendation estimation engine 134 may be configured to use the latent leveling factors to derive a leveling recommendation for one or more combinations of doctors from the first set of doctors and patient types from one of the first set of patient types. Advantageously, the leveling recommendation may have a high likelihood of being applicable to future populations of doctors and/or patient types. Advantageously, the leveling recommendation may be based on latent leveling factors which were previously unknown and/or unknowable. In various implementations, the leveling parameter recommendation estimation engine 134 may be configured to store leveling recommendations for various combinations of doctors and patient types in the leveling parameter recommendation datastore 140.

The leveling parameter doctor datastore 136 may be configured to store historic leveling parameters provided by doctors at some time. The leveling parameter doctor datastore 136 may include a datastore that indexes leveling parameters prescribed in the past by doctor. In some implementations, the historical leveling parameters in the leveling parameter doctor datastore 136 implements a matrix of historic leveling recommendations by doctor. The leveling parameter patient type datastore 138 may be configured to store historic leveling parameters prescribed to different patient types over time. The leveling parameter patient type datastore 138 may include a datastore that indexes leveling recommendations by patient type. In various implementations, the leveling parameter patient type datastore 138 implements a matrix of historic leveling recommendations by patient type.

The leveling parameter recommendation datastore 140 may be configured to store latent leveling factors, such as those latent leveling factors identified and/or derived by the leveling parameter recommendation estimation engine 134.

FIG. 1B shows an example of a system 100B for generating leveling recommendations derived from latent leveling factors. The system 100B may include a patient type identification engine 142, a doctor association engine 144, an estimated leveling parameter recommendation gathering engine 146, a patient datastore 148, a doctor datastore 150, and the leveling parameter recommendation datastore 140. One or more of the modules of the system 100B may be coupled to one another or to modules not explicitly shown.

The patient type identification engine 142 may be configured to gather patient data of one or more patients from the patient datastore 148. The patient type identification engine 142 may further be configured to identify patient types of patients whose information has been gathered. The doctor association engine 144 may be configured to gather from the doctor datastore 148 doctor identifiers of doctors implementing a treatment plan. The doctor association engine 144 may further be configured to associate patient data with a doctor identifier of a doctor implementing a treatment plan.

The estimated leveling parameter recommendation gathering engine 146 may be configured to identify one or more leveling recommendations for a doctor/patient type pair. In some implementations, the leveling recommendations are gathered from the leveling parameter recommendation datastore 140. As noted herein, the leveling parameters may provide latent statistical basis to associate a first set of the historic leveling parameters with a first set of doctors in a first dataset and further providing a basis to associate a second set of historical leveling parameters with a first set of patient types in a second dataset.

As also noted herein, leveling parameter recommendation datastore 140 may be configured to store latent leveling factors, such as those latent leveling factors identified and/or derived by the leveling parameter recommendation estimation engine 134 (see FIG. 1A).

The patent datastore 148 may include a datastore configured to store patient data of a patient. Such patient data may include identifiers of gender, heritage, and/or other background data of a patient as well as identifiers associated with jaw shape/size, arch shape/size, facial characteristics, and/or other physical characteristics. The patient data may form a basis to associate a patient with a patient type. The doctor datastore 150 may be configured to store doctor data of doctors. The doctor data may include identifiers of doctors as well as information about treatment plans that those doctors have implemented in the past.

FIG. 1C shows an example of a method 100C for estimating leveling recommendations using application of historical leveling parameters associated with doctors and application of historical leveling parameters associated with patient types. The method 100C may be executed by one or more modules described herein, including but not limited to the modules of the system 100A shown in FIG. 1A. It is noted the method 100C may include a greater or fewer number of operations than those depicted. It is noted other structures may operate to perform the operations of the method 100C.

At an operation 152, a first dataset including a first set of historic leveling parameters may be gathered. In some implementations, one or more of the first set of the historic leveling parameters is associated with a first set of doctors implementing a treatment plan. In some implementations, the latent factor determination engine 132 may gather from the leveling parameter doctor datastore 136 a first set of historic leveling parameters.

At an operation 154, a second dataset including a second set of historic leveling parameters may be gathered. In various implementations, one or more of the second set of historic leveling parameters is associated with a first set of patient types of patients who have undergone the treatment plan. As noted herein, the latent factor determination engine 132 may gather from the leveling parameter patient type datastore 138 a second set of historic leveling parameters where those historic leveling parameters are associated with a first set of patient types of patients who have undergone a treatment plan.

At an operation 156, a set of latent leveling factors may be derived from the first dataset and the second dataset. In various implementations, one or more of the latent leveling factors provides a latent statistical basis to associate the first set of the historic leveling parameters with the first set of doctors in the first dataset. One or more of the latent leveling factors may also provide a basis to associate the second set of historical leveling parameters with the first set of patient types in the second dataset. As discussed further herein, the leveling parameter recommendation estimation engine 134 may operate to derive a set of latent leveling factors from the first dataset and the second dataset.

At an operation 158, the set of leveling factors may be regularized. At an operation 160, one or more of the latent leveling factors may be removed if they indicate bias. The leveling parameter recommendation estimation engine 134 may operate to implement operations 158 and/or 160.

At an operation 162, the latent leveling factors may be used to derive a leveling

recommendation for one or more combinations of doctors from the first set of doctors and patient types from one of the first set of patient types. The leveling parameter recommendation estimation engine 134 may operate, as discussed further herein, to derive a leveling recommendation for one or more combinations of doctors from the first set of doctors and patient types from one of the first set of patient types.

At an operation 164, a leveling recommendation database may be created using the derived leveling recommendations. As noted herein, the leveling parameter recommendation estimation engine 134 may operate to store one or more derived leveling recommendations in the leveling parameter recommendation datastore 140.

FIG. 1D shows an example of a method 100D for generating leveling recommendations derived from latent leveling factors. The method 100D may be executed by one or more modules described herein, including but not limited to the modules of the system 100B shown in FIG. 1B. It is noted the method 100D may include a greater or fewer number of operations than those depicted. It is noted other structures may operate to perform the operations of the method 100D.

At an operation 170, patient data of a patient is gathered. In some implementations, the patient type identification engine 142 may gather patient data from the patent datastore 148. The patient data may have been manually inputted, may have been crawled through one or more automated agents, or gathered from a larger universe of patient data using various data gathering techniques.

At an operation 172, the patient data is associated with a doctor identifier of a doctor implementing a treatment plan. The doctor association engine 144 may gather from the doctor datastore 148 a doctor identifier of a doctor implementing a treatment plan. At an operation 174, the patient data may be associated with a patient type identifier of a patient type. In some implementations, the patient type identification engine 142 may operate to associate the patient data with a patient type identifier of a patient type. The association may be due to, e.g., gender, heritage, and/or other background data of a patient as well as identifiers associated with jaw shape/size, arch shape/size, facial characteristics, and/or other physical characteristics.

At an operation 176, the doctor identifier and the patient type identifier may be used to identify a leveling recommendation. In some implementations, the leveling recommendation is derived from one or more latent leveling factors. As noted herein, in some implementations, the leveling factors provide a latent statistical basis to associate a first set of the historic leveling parameters with a first set of doctors in a first dataset. The leveling factors may further provide a basis to associate a second set of historical leveling parameters with a first set of patient types in a second dataset.

At an operation 178, instructions to display the leveling recommendation may be provided. In various implementations, the estimated leveling parameter recommendation gathering engine 146 may be configured to translate the leveling recommendation to a representation to be used in orthodontic treatment plans. As an example, in some implementations, the estimated leveling parameter recommendation gathering engine 146 may provide instructions to identify one or more 32-vector representations of leveling recommendations. As another example, the estimated leveling parameter recommendation gathering engine 146 may provide instructions to fabricate shell aligners, such as those used for an incremental orthodontic treatment plan, to implement the leveling recommendation.

FIG. 1E schematically illustrates one variation of a method for providing a leveling recommendation as will be described in greater detail below. In FIG. 1E, the method illustrated may include inputting (e.g., into a computing system) an initial anterior leveling dataset of the patient's anterior teeth 101. The user (e.g., doctor, dentist, technician, etc.) may then input an incisor preference (b_{pref}) regarding a leveling position of lateral incisors 103. The Incisor preference may indicate position of lateral incisors relative to central incisors. An average anterior leveling (μ) dataset may then be derived from a plurality of patients 105, including datasets in a library or database of patient information. Based on this information, the method may then determine a final leveling dataset based on the initial anterior leveling dataset, the incisor preference received from the doctor and the average anterior leveling dataset 107. Determining a final leveling dataset may include latent factor such as country bias, gender bias, doctor/gender bias, etc.). As will be described in greater detail below, any of these methods may use matrix factorization to determine the final leveling dataset. The final leveling dataset may include possible leveling recommendations and a score for each leveling recommendation.

In some embodiments, the final leveling recommendation may be selected from the final leveling dataset 109 (e.g., the set having the highest score from the set of possible leveling recommendations). In some variations, the final leveling recommendation is added to the average anterior leveling dataset 111.

In any of these methods, a posterior leveling dataset may be determined based on the patient's posterior teeth (e.g., determining the dataset by determining the final leveling data set based on the initial anterior leveling dataset, the incisor preference received from the doctor, the average anterior leveling dataset, and the posterior leveling dataset) 113.

Once determined, the final leveling recommendation may be displayed (e.g., to the doctor), from the final leveling dataset 115.

### Anterior Leveling Recommendation System Using Sparse-matrix Factorization

As mentioned above, matrix-factorization via a sparse version of singular value decomposition (SVD) can be deployed in a tooth-leveling recommendation system for anterior positioning of a patient's teeth and for determining arch shape.

When used with dense matrices, singular value decomposition of an m×n matrix M can be is represented as:

$$M = U Σ {V}^{*} \qquad ( 1 )$$

where U is an m×m matrix, 2 is an m x n diagonal scaling matrix, and V* is an n×n matrix. In the context of a recommendation system, M can be considered to be a matrix where each row represents a user and each column represents an item. Cells of the matrix indicate a rating. The decomposition of M can be thought of as identifying a set of n latent factors (equivalent to the principal axes of a PCA) in the columns of V* and the mappings (UΣ) of the original rows onto the latent factors. The matrices are such that Σ is ordered from the most relevant factor to the least. The advantage of SVD is that the number of columns in U, the number of rows in V*, and the number of rows and columns in the scaling matrix Σ can be truncated to obtain a close approximation of M.

In a tooth-leveling recommendation system, this corresponds to finding the L most significant latent factors that represent the ratings of users for each item. This can be represented as factoring the rating matrix as:

$$M = PQ \qquad ( 2 )$$

where M is the |users|×|items| matrix of ratings, P is a |users|×L matrix of users by latent factors, and Q is an L×|items| matrix of the latent factors for each item. M is a sparse matrix and the factorization cannot be performed by SVD. Instead, a parameter adjustment technique can be applied, such as Stochastic Gradient Descent (SGD), to attempt to learn the factorization from the sparse set of ratings.

To learn the factorization of the sparse rating matrix, the matrices P and Q can be initialized with some small values. Then, starting with the first latent factor l=1, iterating through each known rating for product i by user u, rᵤᵢ, and computing the current prediction of the rating:

$${\hat{r}}_{ui} = {\sum }_{k = 1}^{l} {p}_{uk} {q}_{ki} \qquad ( 3 )$$

which is the sum of the user's preferences times the qualities of the item for all latent factors that have been completed so far are currently being worked on, l.

The error between the prediction and the actual rating is:

$${ϵ}_{ui} = {r}_{ui} - {\hat{r}}_{ui} \qquad ( 4 )$$

Finding the best solution for P and Q is equivalent to minimizing the following over the entire set of ratings K:

$${min}_{P , Q} {\sum }_{( u , i ) ∈ 𝒦} {( {r}_{ui} - {\sum }_{k = 1}^{l} {p}_{uk} {q}_{ki} )}^{2} \qquad ( 5 )$$

which can be achieved using SGD by finding the derivative of the error with respect to P and Q:

$$\frac{\partial {e}_{ui}^{2}}{\partial {p}_{ul}} = 2 {e}_{ui} \frac{\partial }{\partial {p}_{ul}} {e}_{ui} \qquad ( 6 )$$

$$= 2 {e}_{ui} \frac{\partial }{\partial {p}_{ul}} ( {r}_{ui} - {\sum }_{k = 1}^{l} {p}_{uk} {q}_{ki} ) \qquad ( 7 )$$

$$= - 2 {e}_{ui} {q}_{li} \qquad ( 8 )$$

$$\frac{\partial {e}_{ui}^{2}}{\partial {q}_{li}} = 2 {e}_{ui} \frac{\partial }{\partial {q}_{li}} {e}_{ui} \qquad ( 9 )$$

$$= 2 {e}_{ui} \frac{\partial }{\partial {q}_{li}} ( {r}_{ui} - {\sum }_{k = 1}^{l} {p}_{uk} {q}_{ki} ) \qquad ( 10 )$$

$$= - 2 {e}_{ui} {p}_{ul} \qquad ( 11 )$$

and by moving P and Q in the opposite direction:

$${p}_{ul} ← {p}_{ul} + γ {e}_{ui} {q}_{li} \qquad ( 12 )$$

$${q}_{li} ← {q}_{li} + γ {e}_{ui} {p}_{ul} \qquad ( 13 )$$

where γ is a learning rate parameter and is typically set low at approximately 0.001.

By iterating over the entire set of ratings several times, the first latent factor, represented in the column vector pu1 and the row vector q1i, can be learned. The error can be minimized using the first latent factor, and the next latent factor l=2 can be learned through the same stochastic gradient descent algorithm. The process can then be repeated until all L factors are learned.

In practice, a latent factor model will have |users|×|items|×L parameters. It is common to have results with over one hundred million parameters. Thus, the model as stated is prone to over fitting. The solution to the over-fitting problem is regularization—penalizing complicated models in some way. The most common approach is to modify Equation 5 by including the [symbol] ₂ norm of P and Q, indicated by |.|.

$${min}_{P , Q} {\sum }_{( u , i ) ∈ 𝒦} {( {r}_{ui} - {\sum }_{k = 1}^{l} {p}_{uk} {q}_{ki} )}^{2} + λ ( {‖ P ‖}^{2} + {‖ Q ‖}^{2} ) \qquad ( 14 )$$

since

$$\frac{\partial }{\partial X} {‖ X ‖}^{2} = \frac{\partial }{\partial X} Tr ( {XX}^{T} ) = 2 X \qquad ( 15 )$$

the update equations become:

$${p}_{ul} ← {p}_{ul} + γ ( {e}_{ui} {q}_{li} - λ {p}_{ul} ) \qquad ( 16 )$$

$${q}_{li} ← {q}_{li} + γ ( {e}_{ui} {p}_{ul} - λ {q}_{li} ) \qquad ( 17 )$$

To remove biases, Equation 3 can be replaced with:

$${\hat{r}}_{ui} = μ + {b}_{u} + {b}_{i} + {\sum }_{k = 1}^{L} {p}_{uk} {q}_{ki} \qquad ( 18 )$$

where μ is the average of all item ratings, bᵢ is the difference between the average rating of item i and μ, and bᵤ is the average over all rᵤᵢ−bᵢ. bᵢ can be thought of as the quality of the item with respect to all other items and bᵤ is how easily user μ is satisfied. A basic predictor, using L=0, is then:

$${{r}^{′}}_{ui} = μ + {b}_{u} + {b}_{i} \qquad ( 19 )$$

indicating that the prediction of user u's rating of item i is the average of all ratings plus the difference in quality of item i as compared to the average plus the user's (lack of) pickiness.

As with the latent factors, the biases can then be regularized by finding the solution to:

$${min}_{{b}_{i} , {b}_{u} , P , Q} {\sum }_{( u , i ) ∈ 𝒦} {( {r}_{ui} - μ - {b}_{u} - {b}_{i} - {\sum }_{k = 1}^{l} {p}_{uk} {q}_{ki} )}^{2} + λ ( {b}_{u}^{2} + {b}_{i}^{2} + {‖ P ‖}^{2} + {‖ Q ‖}^{2} ) \qquad ( 20 )$$

This approach can first compute the average rating, μ, then find the optimal, regularized set of bᵤ and bᵢ before finding the set of latent factors as described above using the update equations:

$${b}_{u} ← {b}_{u} + γ ( {e}_{ui} - λ {b}_{u} ) \qquad ( 21 )$$

$${b}_{i} ← {b}_{i} + γ ( {e}_{ui} - λ {b}_{i} ) \qquad ( 22 )$$

From the standpoint of recommendation systems for tooth-leveling that have been created to-date, there are a number of challenges with recommending a specific leveling:

1) an explicit set of training ratings does not exist, instead there is only an implicit rating of the final, accepted, leveling,

2) the implicit rating is no longer a scalar value, but is instead a vector of levelings, so that the matrix factorization approach as described above no longer applies directly, and

3) each patient only sees a single doctor, so there is only one recommendation per patient.

In the following disclosure, these issues are addressed and a new recommendation system is developed for tooth leveling that considers both the initial arch shape, initial tooth leveling, and learned doctor preferences.

Arch shapes are generally parameterized. The initial arch shape can be parameterized plus the initial leveling in RI and the final leveling in R32, where I is the number of basis vectors needed to represent the concatenation of the parameterized arch shape plus the parameterized initial tooth leveling.

Having parameterized the initial arch shape and leveling in RI and the final leveling in R32 it can be seen how recommendations might be implemented in a tooth leveling recommendation system.

In a typical rating system, a doctor considers a particular item and assigns it a rating, e.g., 1-5 stars. In our case, we only have implicit ratings, a given accepted leveling can be thought of as being rated highly, but we have no examples where a given doctor rates a leveling poorly, except for the initial levelings which are implicitly bad since they are to be corrected. A doctor provides a mapping from the initial arch shape and leveling to the final leveling:

$$f : {ℝ}^{I} \to {ℝ}^{32} \qquad ( 23 )$$

The doctor's mapping can be based on a number of biases plus latent factors. Rather than predicting a doctor's approval for a particular leveling (initial or final), the initial, parameterized patient data can be used to predict a final leveling directly.

Each bias can be represented as an leveling offset and the matrix vector product of a doctor's latent preferences (a vector of length L) can be computed with a matrix consisting of the patient's latent factors (of size 32×L).

The goal becomes the construction of a mapping on a per-doctor/per-patient basis. Unfortunately, a patient only sees a single doctor and has only one example of an initial arch, initial leveling, and final leveling for a given patient. The solution to this problem is to identify groups of patient arches by clustering the data using K-Means, agglomerative clustering, Birch, or some other appropriate algorithm for mapping R^{I} to one of a set of patient types, P. This mapping considers how a given doctor will approach a given type of patient as compared to a different doctor examining a similar patient. Note, for this to be effective, the representation of the initial arch shape should be made in a scale and rotation invariant feature space such as is provided by normalized EFDs.

The leveling that was accepted for a given doctor, d, and patient type, p, can be termed L_{pd}. The leveling recommender will suggest an [symbol] _{pd} that will include the average final tooth leveling across the data set (mu or μ), the leveling requested in the treatment form (b_{pref} with values of Laterals0_5mmMoreGingivalThenCentrals, LateralsLevelWithCentrals, and GingivalMargins), a per-country bias (b_{c}), a per-gender bias (b_{g}), a per-patient type bias (bₚ), and a per-doctor/per-gender bias (b_{dg}) plus a set of latent factors for each patient type, Aₚ, and the particular doctors preferences for those factors, D_{d}:

$$\hat{L} = μ + {b}_{pref} + {b}_{c} + {b}_{g} + {b}_{p} + {b}_{dg} + {\sum }_{l = 1}^{L} {d}_{d}^{l} {a}_{p}^{l} \qquad ( 24 )$$

where d_{d}^{l} is the 1-th latent factor for doctor d and aₚ^{l} is the 1-th latent vector for patient type p.

To compute the biases and the latent factors, initialize all [symbol] _{pd} can be initialized to =0. The error residual is then:

$${R}_{pd} = {L}_{pd} - {\hat{L}}_{pd} \qquad ( 25 )$$

$$= {L}_{pd} - 0 \qquad ( 26 )$$

$$= {L}_{pd} \qquad ( 27 )$$

To find μ, the following can be computed:

$$μ = \frac{1}{\vert \vert} {\sum }_{j ∈} {R}_{j} \qquad ( 28 )$$

and the residual can be updated:

$${R}_{j} ← {R}_{j} - μ : ∀ j ∈ \qquad ( 29 )$$

To find the bias given the prescription form, a regularized b_{pref} can be assumed as a Gaussian prior around 0 with N initial observations. The preferences bias is then:

$${b}_{pref} = \frac{1}{\vert {pref}^{} \vert + N} {R}_{j} \qquad ( 30 )$$

where K^{pref} is the subset of accepted leveling with a prescription form leveling preference, pref. Again, the residual is updated as:

$${R}_{j} ← {R}_{j} - {b}_{pref} ( j ) ; ∀ j ∈ \qquad ( 31 )$$

where b_{pref} (j) is the preference bias for the preference exhibited in case j.

Similarly, a regularized b_{c} with a Gaussian prior around 0 with N initial observations can be found as:

$${b}_{c} = \frac{1}{\vert {c}^{} \vert + N} {R}_{j} \qquad ( 32 )$$

where K^{c} is the subset of accepted leveling from country, c. Again, the residual is updated as:

$${R}_{j} ← {R}_{j} - {b}_{c} ( j ) ; ∀ j ∈ \qquad ( 33 )$$

where b_{c} (j) is the country bias for the country in which case j occurred.

A regularized b_{g} is found as:

$${b}_{g} = \frac{1}{\vert {g}^{} \vert + N} {R}_{j} \qquad ( 34 )$$

where K^{g} is the subset of accepted leveling for each gender, g. The residual is updated as:

$${R}_{j} ← {R}_{j} - {b}_{g} ( j ) ; ∀ j ∈ \qquad ( 35 )$$

where b_{g} (j) is the gender bias for case j.

The remaining biases can be found in a similar manner. Once the biases have been computed, the residual error remains which can be captured through a modified matrix factorization where the D_{d} is the doctor's preferences for the latent factors represented in the 3-dimensional tensor Aₚ which has dimensions L×P×32, where L is the number of latent factors, P is the number of patient types, and 32 represents the number of possible teeth in the leveling. In this scenario, the latent factors are per-patient class vectors of leveling deltas and a doctor's per-latent factor preference can be computed for this delta, so that the final prediction of the tooth leveling is as given in Equation 24, where aₚ^{l} represents the l-th latent factor for patient class p, and d_{d}^{l} is doctor d's preference for the l-th latent factor.

There is no learning involved in computing μ or the various biases, however, both aₚ^{l} and d_{d}^{l} can be learned such as stochastic gradient descent, where the update rules are as follows:

$$\frac{\partial }{\partial {d}_{d}} {R}_{pd}^{T} {R}_{pd} = - 2 {R}_{pd}^{T} {a}_{p} \qquad ( 36 )$$

$$\frac{\partial }{\partial {a}_{p}} {R}_{pd}^{T} {R}_{pd} = - 2 {d}_{pd} {R}_{pd} \qquad ( 37 )$$

resulting in:

$${d}_{d}^{l} ← {d}_{d}^{l} + γ {R}_{pd}^{T} {a}_{p}^{l} \qquad ( 38 )$$

$${a}_{p}^{l} ← {a}_{p}^{l} + γ {d}_{d}^{l} {R}_{pd} \qquad ( 39 )$$

Having normalized the levelings in the dataset, the Anterior Leveling model and its individual subcomponents can be used to predict the doctor's accepted anterior leveling preferences based on the initial leveling, arch shape, and prescription form. As described in the previous sections, the initial arch shape is represented via the first principal components of a PCA of the Elliptic Fourier Descriptor representation of the arch points. The first 12 components of the normalized EFD can be used which captures essentially 100% of the explained variance in the data.

The input feature data can then be clustered into a plurality of patient types (e.g., 500 different patient types) using a batched K-means method. The model was then fit to the data, as described below.

Table 1 presents the results of running various portions of the anterior leveling model given in Equation 24. The metric used is the [symbol] ₂ norm of the difference between the prediction and the doctor's anterior leveling.

TABLE 1
Various models for anterior tooth leveling prediction
and their scores as compared to the doctor's accepted levelings.

| Model | Score |
|---|---|
| L_{pd} = X₁ | 788.41 |
| L_{pd} = 0 | 75.41 |
| L_{pd} = μ | 70.35 |
| L_{pd} = μ + b_{pref} | 6.84 |
| L_{pd} = μ + b_{pref} + b_{g} | 6.84 |
| L_{pd} = μ + b_{pref} + b_{g} + b_{c} | 6.83 |
| L_{pd} = μ + b_{pref} + b_{g} + b_{c} + bₚ | 6.82 |
| L_{pd} = μ + b_{pref} + b_{g} + b_{c} + bₚ + b_{dg} | 6.31 |
| L_{pd} = μ + b_{pref} + b_{g} + b_{c} + bₚ + b_{dg} + | 5.92 |
| Σₗ₌₁^{L} d_{d}¹aₚ¹ | |
| L_{pd} = μ + b_{g} | 70.35 |
| L_{pd} = μ + b_{c} | 69.78 |
| L_{pd} = μ + bₚ | 70.18 |
| L_{pd} = μ + b_{dg} | 35.45 |
| L_{pd} = μ + Σₗ₌₁^{L} d_{d}¹aₚ¹ | 16.89 |

As may be seen from the table able, when determining a final leveling dataset based on the patient's initial anterior leveling dataset using the methods described herein, it was surprisingly found that an average anterior leveling (μ) dataset derived from a plurality of patients provided a dramatic increase in the predictive score (e.g., line three in table 1, above). More surprisingly, the addition of the doctor's incisor preference (b_{pref}) as received from the doctor regarding a leveling position of lateral incisors resulted in a much greater improvement in the predictive score (e.g., see line four in table 1, above). Other latent factors, including in particularly doctor/gender bias (b_{dg}) were also significant, but the use of at least μ and b_{pref} provides a substantial increase in predictive strength.

Arch Shape Recommendation using Sparse-matrix Factorization

The same techniques as described above with respect to the anterior leveling recommendation system can be applied for an arch shape recommendation system.

From the standpoint of a recommendation system, there are a number of challenges with recommending an arch shape:

1) explicit set of training ratings does not exist, instead there is only an implicit rating of the final arch shape,

2) each patient only sees a single doctor, so there is only one recommendation per patient, and

3) the implicit rating is no longer a scalar value, but is instead a shape.

In the following disclosure, these issues are addressed and a new recommendation system is developed for arch shapes that considers both the initial arch shape and learned doctor preferences.

Arch shapes are generally parameterized. Both the initial and final (doctor-approved) arch positions can be parameterized for a patient using RI and RF respectively, where I and F are the number of basis vectors used to represent the initial and final arch shapes.

Having parameterized the initial and final arch shapes in RI and RF it can be seen how recommendations might be implemented.

A doctor can provide a function mapping the initial arch shape to the final:

$$f : {ℝ}^{I} \to {ℝ}^{F} \qquad ( 40 )$$

In matrix notation, if it is assumed that the initial arch shape is a column vector, x, and the final arch shape is a column vector, y, then:

$$y = Mx \qquad ( 41 )$$

Where M is a F×I transform matrix provided implicitly by the doctor.

The goal becomes constructing M on a per-doctor/per-patient basis, M_{dp}. Unfortunately, while this addresses the issue of a rating being a shape, it does not address the issue of a patient only seeing a single doctor and only having one example of an initial and final arch for a given patient.

The solution to this problem is to identify groups of patient arches by clustering the data using K-Means, agglomerative clustering, Birch, or some other appropriate algorithm for mapping R^{I} to one of a set of patient types, P. This mapping considers how a given doctor will approach a given type of patient as compared to a different doctor examining a similar patient. Note, for this to be effective, the representation of the initial arch shape should be made in a scale and rotation invariant feature space such as is provided by EFDs.

While the issue of having multiple representations of arch types has been addressed, there remains a need to consider which factors are doctor-specific preferences and which are global factors for a given arch shape. To achieve this, M_{dp} can be factored as

$${M}_{dp} = {D}_{d} {A}_{p} \qquad ( 42 )$$

Where Aₚ is a F×I matrix that maps orthodontic practice for initial arch shape p to the final arch shape in R^{F} and D_{d} is a F×F matrix representing doctor d's preferences with respect to the final arch parameters. With this factorization, the relationship between the initial and suggested final arch shapes can be written as:

$$\hat{y} = {D}_{d} {A}_{p} x \qquad ( 43 )$$

As with any rating system, the system can be extended to remove biases to allow for multiple latent factors:

$${\hat{y}}_{dp} = ( μ + {b}_{d} + {b}_{p} + {\sum }_{l = 1}^{L} {D}_{d}^{l} {A}_{p}^{l} ) {x}_{p} \qquad ( 44 )$$

Where d_{d}^{l} and aₚ^{l} are 1-th latent factor matrices.

Note that the system no longer fits into the SVD model of a single rating matrix being factored into a matrix of items with respect to their latent factors and a matrix of users and their preferences for latent factors. That said, the same least-squares approach used to estimate the factorization from a sparse set of ratings can still be used to determine the parameters of the arch recommendation system by utilizing SGD and a set of K of (xₚ, y_{dp}) vectors. Given that:

$${ϵ}_{dp}^{2} = {e}_{dp}^{T} {e}_{dp} = {( {y}_{dp} - {\hat{y}}_{dp} )}^{T} ( {y}_{dp} - {\hat{y}}_{dp} ) \qquad ( 45 )$$

the goal is to minimize the error of the set by finding:

$${min}_{μ , {b}_{d} , {b}_{p} , {D}_{d} , {A}_{p}} {e}_{dp}^{T} {e}_{dp} \qquad ( 46 )$$

which can be found by taking the partial derivative of Equation 45 with respect to μ, b_{d}, bₚ, D_{d}, and Aₚ. When regularization is used to attempt to minimize the complexity of the parameters beyond the initial expectation that they be the identity matrix right padded with zero as necessary, I.

While the model above is mathematically very appealing, it has a large number of parameters which are not easy to learn (e.g., success is strongly dependent on the learning rate). In a second variant of the arch recommender, a clustering technique is again used to group the patients into a set of arch types, but instead of attempting to predict a transform matrix from the initial to accepted arch shape, the accepted arch shape is predicted directly based on latent factors. The model computes a bias, μ in R^{F} by computing the average accepted arch shape in the first F dimensions of the PCA-computed space used above.

$$μ = \frac{1}{\vert \vert} {y}_{j} - {x}_{j} \qquad ( 47 )$$

Since u is the average difference between all accepted arch shapes and the initial arch shapes, yⱼ=xⱼ+μ is not expected to be an accurate approximation of the accepted arch shape, but it may normalize the data. To better approximate the accepted arch shape, biases can be added for both the patient-class and the doctor. The patient-class bias can be defined as

$${b}_{p} = \frac{1}{\vert {p}^{} \vert} {y}_{j} - {x}_{j} - μ \qquad ( 48 )$$

where K^{p} is the subset of the training arch shapes where the patient class is p. Note that this defines bₚ as the average residual of the accepted arches of class p and the average of all accepted arches, μ. Similarly, the doctor bias can be defined as:

$${b}_{d} = \frac{1}{\vert {d}^{} \vert} {y}_{j} - {x}_{j} - μ - {b}_{p} \qquad ( 49 )$$

where K^{d} is the subset of the training arch shapes where the doctor is d. This defines the doctor bias as being the average of the residual of the accepted arches with the average arch and the patient-class bias removed.

Now that the biases have been computed, a set of L latent factors can be utilized to describe the remaining residual. In this scenario, the latent factors are per-patient class vectors of arch shape deltas and we compute a doctor's per-latent factor preference for this delta, so that our final prediction of the arch shape is:

$${\hat{y}}_{dp} = {x}_{dp} + μ + {b}_{p} + {b}_{d} + {\sum }_{l = 1}^{L} {a}_{p}^{l} {d}_{d}^{l} \qquad ( 50 )$$

where aₚ^{l} represents the 1-th latent factor for patient class p, and d_{d}^{l} is doctor d's preference for the 1-th latent factor.

In both variants described above, it is possible to weight the importance of different components of the arch shape when learning. One approach to this weighting is to consider the amount explained by each basis vector of the PCA. If v is a vector containing the normalized explained variance of the components of the PCA, then

$$w = \frac{F}{{\sum }_{j = 1}^{F} {v}_{j}} v \qquad ( 51 )$$

where F is the number of components of the PCA that are being used.

Both variants described above were tested, but the second variant performed better and the results below are from that model. The process of learning was as follows:

1) Estimate initial arch shapes using the set of crown center x and y coordinates as a proxy for real information.

2) Compute the 20th degree elliptic Fourier descriptors for the initial arch shapes, storing the results in X

3) Compute the 20th degree elliptic Fourier descriptors for the accepted arch shapes, storing the results in Y

4) Compute the normalized version of the initial arch shapes as Xn

5) Cluster the normalized arch shapes, Xn into 1,000 patient types using mini-batch K-means, storing the classes in xclass

6) Fit a PCA of X, computing the fit on X and Y as Xp and Yp respectively

7) Truncate the number of columns of columns in Xₚ and Yₚ to capture 99% of the variance in the PCA

8) Compute μ

9) Compute bp and bd

10) Learn the L=10 latent factors as described above

The learning process substantially reduces the difference between the predicted arch shape and the accepted arch shape as compared to the initial arch.

Posterior Leveling Recommendation System using Classifier Theory

Unlike anterior leveling in which aesthetic preferences are a primary concern, posterior leveling is driven primarily by clinical factors. The only strong per-doctor factor in posterior leveling is whether the doctor provided any posterior leveling at all. Rather than building a collaborative filtering model as was created for the anterior teeth as described above, the posterior leveling model of the present disclosure will be based primarily on the clinical factors as learned from the data.

The model will be heavily based on classifier theory where the two classes of interest are the “initial” leveling and the doctor's “accepted” leveling. In classifier theory, the Naïve Bayes Classifier relies upon Bayes' Theorem to predict whether a given set of observed variables correspond most closely to one class or another. Consider a classification problem with N classes, C₁, . . . , C_{N}. Given a set of M observed variables, {X₁, . . . , X_{M}}, the goal is to predict the probability of each class Cₙ given X. From Bayes' Theorem, it is known that:

$$p ( {C}_{n} \vert X ) = \frac{p ( X \vert {C}_{n} ) p ( {C}_{n} )}{p ( X )} \qquad ( 52 )$$

Since p(X) is a constant, it can be noted that:

$$p ( {C}_{n} \vert X ) ∝ p ( X \vert {C}_{n} ) p ( {C}_{n} ) \qquad ( 53 )$$

where p(Cn) is the prior probability of class n.

Considered in this way, an approach for comparing the probability that a given set of observed variables belongs to a given class as opposed to another class can be implemented by computing arg maxₙ p(Cₙ|X). The one remaining question is how to determine p(Cₙ|X), particularly when X has a large number of variables. Unless the variables have been chosen carefully, they are almost certainly dependent upon each other. However, the Naïve Bayes assumption is that they are all independent, so:

$$p ( X \vert {C}_{n} ) = {\prod }_{m = 1}^{M} p ( {X}_{m} \vert {C}_{n} ) \qquad ( 54 )$$

Substituting Equation 54 into Equation 53 gives the overall probability of a particular class given a set of observed values, X,

$$p ( {C}_{n} \vert X ) ∝ p ( {C}_{n} ) {\prod }_{m = 1}^{M} p ( {X}_{m} \vert {C}_{n} ) \qquad ( 55 )$$

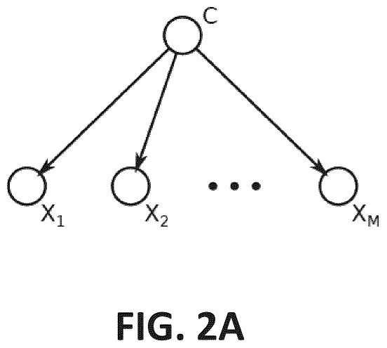

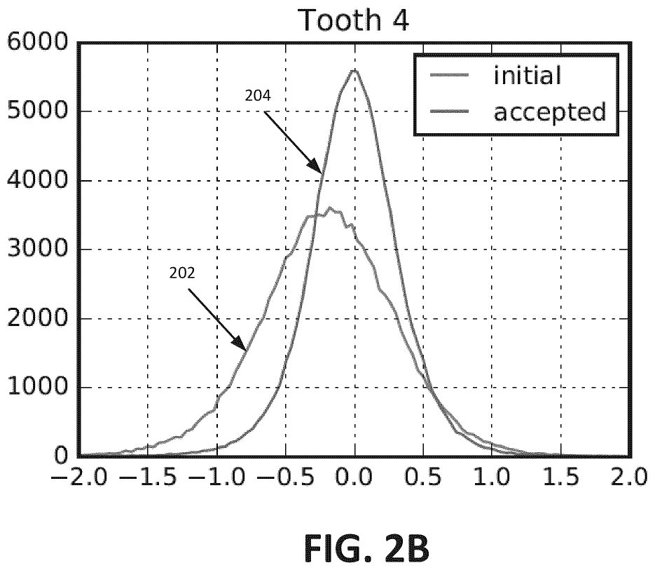

which can be represented as the graphical model in FIG. 2A.

To train the classifier, p(Cn|X) can be empirically computed for each variable, m, and each class, n. A number of approaches can be used for estimating the probability density function from a set of observed data, e.g., Gaussian Kernel Density Estimation. In this method

$$p ( x ) = \frac{1}{N} {\sum }_{i = 1}^{N} K ( x - {x}_{i} ) \qquad ( 56 )$$

where K (·) is some kernel function, in this case Gaussian, xi is the set of observed values for x, and p(x) is the probability of some new point x given the observed values. The “width” of the Kernel function is generally estimated using either Scott's rule or Silverman's Rule.

One challenge of using Gaussian Kernel Density Estimation in practice is that each probability evaluation involves summing over a large number of Gaussian Kernels. In practice, it is more efficient to compute and store a piece-wise interpolation function that approximates the Gaussian KDE.

For scoring potential levelings, it is difficult to consider a leveling and determine whether it comes from the class of “accepted” levelings or “rejected” levelings in part because there exists no sample of considered, but rejected levelings. Instead, consideration must be based on “initial” levelings which may have significant overlap with the class of accepted levelings. Due to this constraint, the probability that a given leveling would be accepted must be determined.

One of the primary factors for consideration in the model is the absolute leveling of each tooth. FIG. 2B shows an example of the initial distribution of levelings for tooth 4 (line 202) and the accepted distribution of leveling (line 204). The Figure shows that, while the distributions overlap, the accepted position has a mean centered on 0, and has less variance around this value than does the initial position. The distributions of all 32 teeth follow this same pattern, though with some variation on the mean. If this histogram were converted to a probability density function, it would be more tightly peaked than a normal distribution with the same variance. This is different from the initial distribution which is very comparable to a normal distribution.

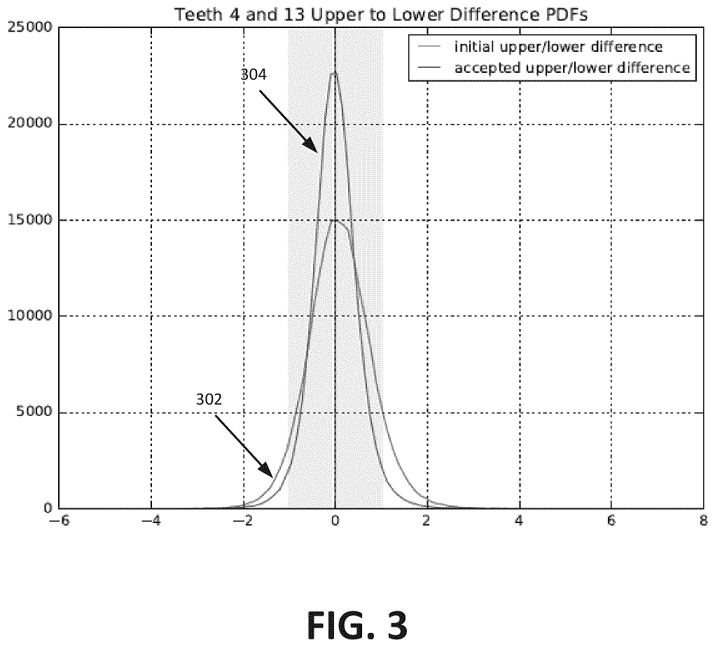

Another factor in the quality of the leveling is the distance between the upper posterior teeth and the lower. In most cases, this centered around 0, but in some cases the mean of both the initial and the accepted difference is non-zero. For this feature, the difference in leveling is computed between the upper posterior teeth and their corresponding lower teeth. Additionally, facial symmetry can be taken advantage of to reduce the number of factors by considering each posterior tooth on the left side of the mouth with its corresponding tooth on the right side of the mouth. An example of this can be seen in FIG. 3 where both the initial (line 302) and accepted (line 304 distributions are centered around 0, but the accepted distribution is more strongly peaked, indicating some movement by doctors toward this position. Note, the shading indicates the 95% confidence interval for the accepted leveling.

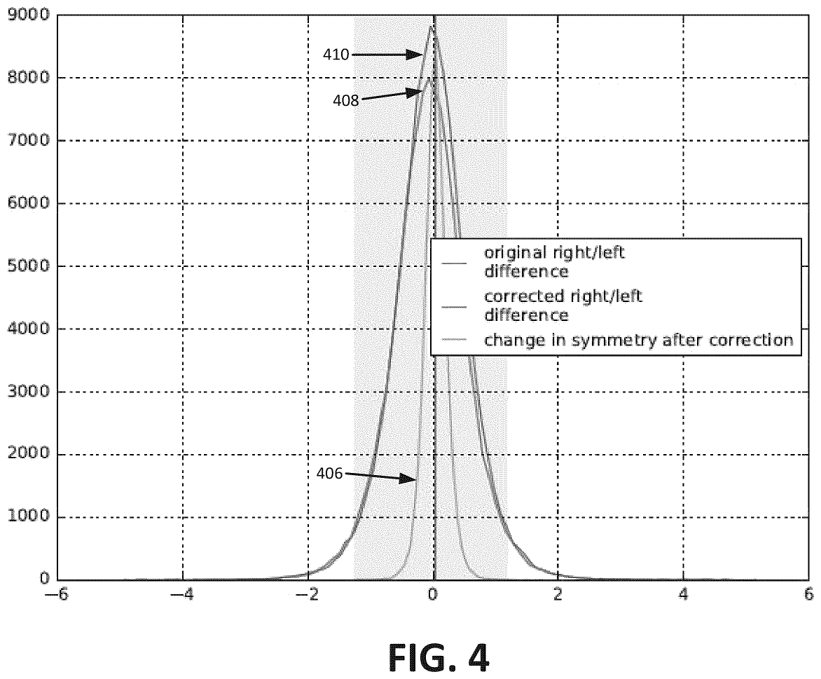

Symmetry is a very important factor when working with teeth. FIG. 4 considers the distribution around the symmetry of the accepted result. For any given patient, this considers the leveling of the teeth as compared to the leveling of the tooth on the opposite side of the mouth. In FIG. 4, this is seen in the change distribution line 406, which shows the change in symmetry after correction from the original distribution line 408 to the corrected distribution line 410.

In the previous section, left/right facial symmetry were considered. This feature considers the change in facial symmetry from the initial to the accepted distribution. In most cases, this is very tightly centered around 0, suggesting that the doctor is attempting to maintain the same facial symmetry differences as existed initially.

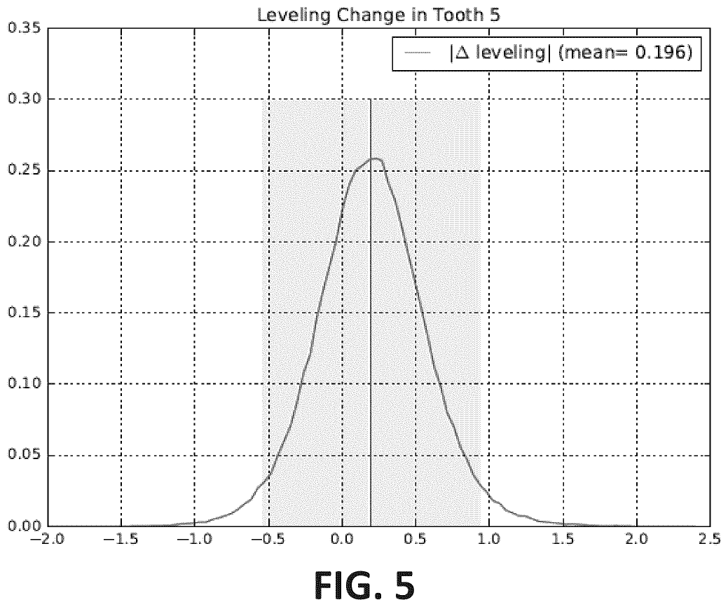

Finally, the total difference between the accepted and initial levelings of each tooth can be considered. By considering how much change is typically seen per-case for each tooth, it can be determined whether a potential leveling recommendation is probable or even possible given the initial leveling. FIG. 5 shows a sample distribution over the change in leveling seen from initial to accepted in tooth 5.

In a normal Naïve Bayes classifier, the probabilities of each factor would be multiplied together, along with the class probability, to obtain an overall probability of seeing the factors given the class. Alternatively, for a more stable evaluation, the logarithm of the probabilities of each factor could be summed. Whichever class had the higher product (or sum) would be the class best represented by the data.

In this disclosure, the main concern is the raw probability of the data given the accepted class, so strict conformance to the Naïve Bayes classifier is not necessary. Instead, a normalized score can be used which allows the comparison of different patients even when they don't have the same set of teeth (and therefore don't have the same set of factors available for calculation). The score for a proposed leveling Y, given an initial leveling X can be computed as:

$$score ( Y \vert X ) = \frac{1}{\vert 𝒫 \vert} {\sum }_{i ∈ 𝒫} log p ( {Y}_{i} ) + \frac{1}{\vert 𝒥 \vert} {\sum }_{i , j ∈ 𝒥} log p ( {Y}_{i} - {Y}_{j} ) + \frac{1}{\vert 𝒮 \vert} {\sum }_{i , j ∈ 𝒮} log p ( {Y}_{i} , {Y}_{j} \vert {X}_{i} , {X}_{j} ) + \frac{1}{\vert 𝒮 \vert} {\sum }_{i , j ∈ 𝒮} log p ( {Y}_{i} - {Y}_{j} ) + \frac{1}{\vert 𝒫 \vert} {\sum }_{i ∈ 𝒫} log p ( {Y}_{i} - {X}_{i} ) \qquad ( 7 )$$

where P is the set of posterior teeth, J is a set of tuples of the posterior teeth in the upper jaw and their corresponding lower tooth, and S is a set of tuples of the right teeth and their corresponding, symmetric tooth on the left.

Equation 57 can be used to score any arbitrary posterior leveling recommendation, including the recommendation of leaving the leveling at its initial position, score (X|X), which has the scoring advantage of not having changed its values and so having high values for log p(Yi, Yj|Xi, Xj) and log p(Yi−Xi). This requires any changes that are made in the leveling to improve the other factors in the model.

Another possible leveling recommendation is to use the most probable leveling across the model, Yi, =arg maxi p (Yi); Vi. While this model will score high in terms of log p(Yi), log p(Yi−Yj), and log p(Yi−Xi), it may incur a significant change penalty from the remaining terms, possibly setting the score to −inf. Ideally, a posterior leveling can be identified that maximizes the score. There are a number of optimization techniques can be used, including line search Newton Conjugate Gradient method, BFGS, Nelder-Mead, the Powell method, and Sequential Least Squares Quadratic Programming (SLSQP). All of these methods, with the exception of the Powell Method, make use of either the exact or an estimate of the Jacobian, and the Newton Conjugate Gradient method requires the Hessian. Experimentally it has been found that the SLSQP method performs best in terms of both speed and optimization.

As described above, the Naïve Bayes Classifier model assumes independence between all of the variables Xₘ in order to factor the conditional probability p(X|Cₙ). In practice, this assumption of independence works reasonably well, often outperforming other state of the art classification methods. However, the independence assumption is often a limiting factor in the accuracy, and therefore performance, of the model. A more principled approach would consider the complete set of conditional probabilities between all of the various observed random variables in X. However, with even a relatively small number of random variables, this becomes intractable. The tree augmented Naïve Bayes algorithm attempts to address these problems by identifying the random variables with the greatest degree of mutual information and then models each variable as being dependent on, at most, one other random variable. The dependence relationships for the new model can then be represented by a tree.

The TAN model begins by constructing an M×M matrix, I where the pair-wise mutual information of the random variables will be stored:

$${I}_{i , j} = Mutual Information ( {X}_{i} , {X}_{j} ) = I ( {X}_{i} ; {X}_{j} ) \qquad ( 58 )$$

where:

$$I ( X ; Y ) = {\int }_{Y} {\int }_{X} p ( x , y ) log ( \frac{p ( x , y )}{p ( x ) p ( y )} ) dx dy , \qquad ( 59 )$$

which can be approximated as:

$$I ( X ; Y ) = {\sum }_{y ∈ Y} {\sum }_{x ∈ X} p ( x , y ) log ( \frac{p ( x , y )}{p ( x ) p ( y )} ) , \qquad ( 60 )$$

when using the approximate Gaussian kernel density estimations.

I can then be used as the weights of a fully connected graph, G, which represents the degree of dependence between the variables. G can be converted into a tree, T, by finding the maximum-weighted spanning tree, using an algorithm such as Dijkstra's or Prim's, then selecting an arbitrary node to be the root node of the tree.

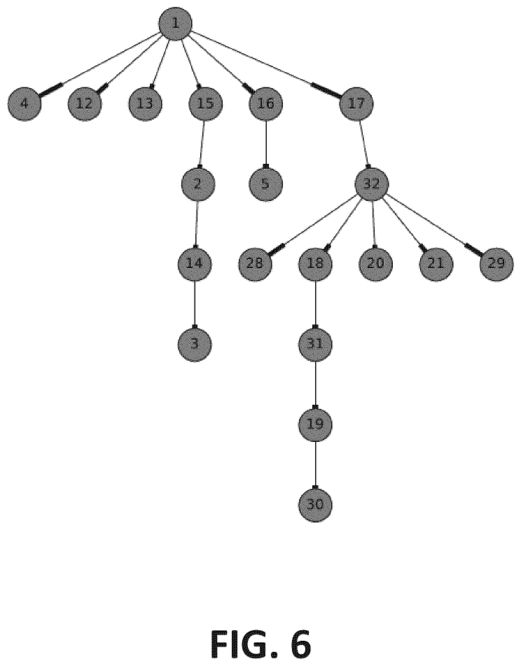

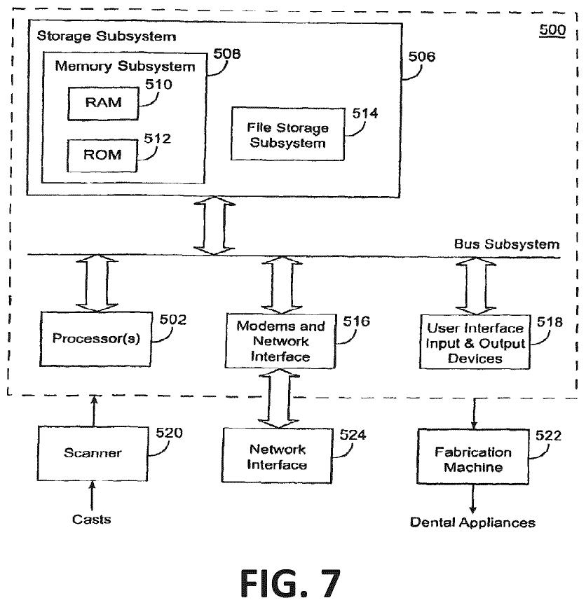

Once constructed, T can be used as a graphical model representing the dependency between variables. Consider the following example of a tree constructed from a subset of variables from the per-tooth change in leveling technique described above. With 20 posterior teeth, there are 20 factors in this model (M=20). The pairwise mutual information can be used to construct the tree shown in FIG. 6.

Since the data does not support the idea of doctor preferences, the posterior model was used to determine a best practice recommendation. For performance reasons, an approximation of the Gaussian KDE was used for probability density estimation. For each probability model of the form p(X|A), a 200-point piecewise linear interpolation function was used over the range of the function which resulted in a substantial performance improvement over computing the Gaussian KDE. To create “optimal” posterior levelings, the Sequential Least Squares Quadratic Programming optimizer built into Python's SciPy module was used with an initial solution of the initial teeth leveling.

As has been previously discussed, a doctor's decisions about posterior leveling appear to be based less on aesthetics and more on clinical factors. Overall, the model's score of the original levelings (which is of course not penalized by moving any teeth) indicated an average score of −1.30 which implies that the original levelings are relatively improbable as accepted final levelings. The doctor's accepted levelings had an average score of −0.87 which indicates a better fit with the model.

Using the Sequential Least Squares Quadratic Programming optimization, leveling were found with an average score of 0.45 which is a significant improvement in the fit with the model (recall that the scores are an average log of the probability of the features).

60% of the accepted levelings had a better score than the initial levelings and 89% of the optimal levelings were better than the accepted levelings. It is likely that more of the accepted levelings would have a better score except that the initial levelings received a bonus (of sorts) by not moving any of the teeth (high log p(Yi−Xi)).

To get a better sense of the strength of the Naive Bayes scorer, two scorers were trained, one using the initial data and one using the accepted data. To avoid having the initial scorer strongly penalize any movement, the features that considered the difference in position between the initial and accepted positions were removed. Once the two scorers were trained, a classification score was computed of the form:

$${Score}_{total} = {Score}_{accept} ( X , Y ) - {Score}_{init} ( X , Y ) . \qquad ( 61 )$$

This was then run against the initial and accepted positions to see if the accepted positions could be correctly identified from the model.

In a similar manner, the Tree Augmented Naïve Bayes model was also tested. In theory, this model which captures some of the dependencies, rather than assuming independence, should have better performance.

By separating the anterior and posterior leveling problems, the present disclosure has identified a pair of algorithms for making recommendations regarding tooth leveling. In general, these algorithms perform very well, capturing doctor's aesthetic preferences for anterior teeth and general clinical best practices for posterior teeth.

In considering the anterior teeth leveling model, a number of observations can be made. First, the model itself does not necessarily have enough information to capture all of the variance in teeth-types. For example, a patient with chipped teeth may have leveling needs that are different than a standard patient, even when the doctor's leveling preference is considered. Second, the simple model of predicting an anterior leveling based solely on the mean plus the doctor's indicated preference on the prescription form captures the almost all of the information regarding the doctor's accepted anterior leveling. Similarly, the latent model without knowledge of the doctor's preference captures a large fraction of the information with an error of 16.89. This suggests that the doctor's general preferences can be learned from the data alone and compares favorably to a model in which the mean is added to the doctor's stated generic preference for tooth leveling which has an error of 10.69.

In general, it is suggested to truncate the model to the mean plus the doctor's leveling preference as stated in the prescription form. Moreover, gender, country, and patient-type have little effect on the error of the model and while doctor preference per patient-type does have an effect, it is likely to be produced primarily by over fitting the parameters.

The posterior leveling model allows suggesting and scoring a variety of levelings.

The posterior leveling model currently relies on a Naïve Bayes assumption.

FIG. 7 is a simplified block diagram of a data processing system 500. Data processing system 500 typically includes at least one processor 502 which communicates with a number of peripheral devices over bus subsystem 504. These peripheral devices typically include a storage subsystem 506 (memory subsystem 508 and file storage subsystem 514), a set of user interface input and output devices 518, and an interface to outside networks 516, including the public switched telephone network. This interface is shown schematically as “Modems and Network Interface” block 516, and is coupled to corresponding interface devices in other data processing systems over communication network interface 524. Data processing system 500 may include a terminal or a low-end personal computer or a high-end personal computer, workstation or mainframe.

The user interface input devices typically include a keyboard and may further include a pointing device and a scanner. The pointing device may be an indirect pointing device such as a mouse, trackball, touchpad, or graphics tablet, or a direct pointing device such as a touchscreen incorporated into the display. Other types of user interface input devices, such as voice recognition systems, may be used.

User interface output devices may include a printer and a display subsystem, which includes a display controller and a display device coupled to the controller. The display device may be a cathode ray tube (CRT), a flat-panel device such as a liquid crystal display (LCD), or a projection device. The display subsystem may also provide nonvisual display such as audio output.

Storage subsystem 506 maintains the basic programming and data constructs that provide the functionality of the present invention. The software modules discussed above are typically stored in storage subsystem 506. Storage subsystem 506 typically comprises memory subsystem 508 and file storage subsystem 514.

Memory subsystem 508 typically includes a number of memories including a main random access memory (RAM) 510 for storage of instructions and data during program execution and a read only memory (ROM) 512 in which fixed instructions are stored. In the case of Macintosh-compatible personal computers the ROM would include portions of the operating system; in the case of IBM-compatible personal computers, this would include the BIOS (basic input/output system).

File storage subsystem 514 provides persistent (nonvolatile) storage for program and data files, and typically includes at least one hard disk drive and at least one floppy disk drive (with associated removable media). There may also be other devices such as a CD-ROM drive and optical drives (all with their associated removable media). Additionally, the system may include drives of the type with removable media cartridges. The removable media cartridges may, for example be hard disk cartridges, such as those marketed by Syquest and others, and flexible disk cartridges, such as those marketed by Iomega. One or more of the drives may be located at a remote location, such as in a server on a local area network or at a site on the Internet's World Wide Web.

In this context, the term “bus subsystem” is used generically so as to include any mechanism for letting the various components and subsystems communicate with each other as intended. With the exception of the input devices and the display, the other components need not be at the same physical location. Thus, for example, portions of the file storage system could be connected over various local-area or wide-area network media, including telephone lines. Similarly, the input devices and display need not be at the same location as the processor, although it is anticipated that the present invention will most often be implemented in the context of PCS and workstations.

Bus subsystem 504 is shown schematically as a single bus, but a typical system has a number of buses such as a local bus and one or more expansion buses (e.g., ADB, SCSI, ISA, EISA, MCA, NuBus, or PCI), as well as serial and parallel ports. Network connections are usually established through a device such as a network adapter on one of these expansion buses or a modem on a serial port. The client computer may be a desktop system or a portable system.

Scanner 520 is responsible for scanning casts of the patient's teeth obtained either from the patient or from an orthodontist and providing the scanned digital data set information to data processing system 500 for further processing. In a distributed environment, scanner 520 may be located at a remote location and communicate scanned digital data set information to data processing system 500 over network interface 524.

Fabrication machine 522 fabricates dental appliances based on intermediate and final data set information received from data processing system 500. In a distributed environment, fabrication machine 522 may be located at a remote location and receive data set information from data processing system 500 over network interface 524.

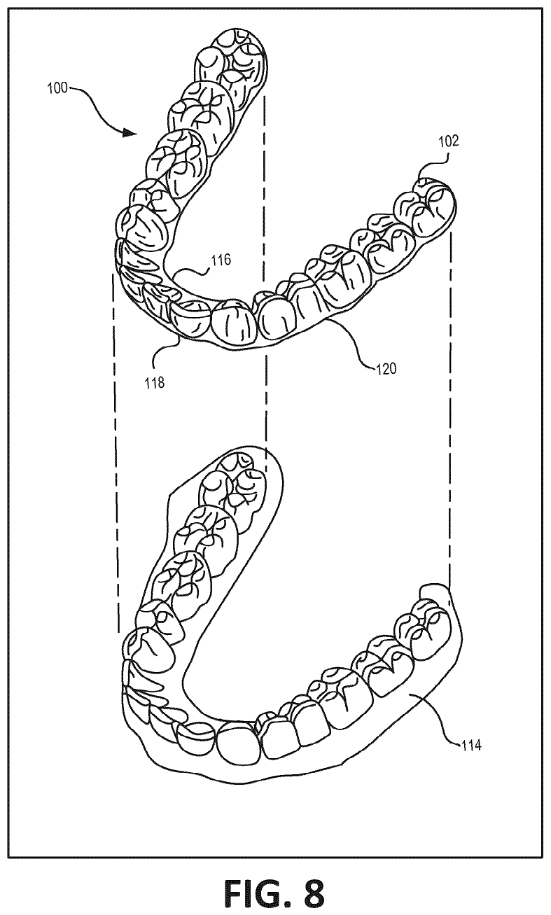

FIG. 8 shows the lower jaw 114 of a patient together with an orthodontic aligner 100, which can be designed according to the methods and techniques described above. The orthodontic aligner 100 is removable and replaceable over the teeth. In some embodiments, orthodontic aligner 100 is one of a plurality of incremental position adjustment appliances.

The orthodontic aligner 100 may comprise a polymeric concave trough 102 having an inner cavity 120, a proximal edge 116, and a distal edge 118. The inner cavity is shaped to receive and resiliently reposition teeth from one tooth arrangement to a successive tooth arrangement. In some embodiments, the polymeric concave trough 102 fits over all teeth present in the upper jaw (not depicted) or lower jaw 114.

Various alternatives, modifications, and equivalents may be used in lieu of the above components. Although the final position of the teeth may be determined using computer-aided techniques, a user may move the teeth into their final positions by independently manipulating one or more teeth while satisfying the constraints of the prescription.

Additionally, the techniques described here may be implemented in hardware or software, or a combination of the two. The techniques may be implemented in computer programs executing on programmable computers that each includes a processor, a storage medium readable by the processor (including volatile and nonvolatile memory and/or storage elements), and suitable input and output devices. Program code is applied to data entered using an input device to perform the functions described and to generate output information. The output information is applied to one or more output devices.

Each program can be implemented in a high level procedural or object-oriented programming language to operate in conjunction with a computer system. However, the programs can be implemented in assembly or machine language, if desired. In any case, the language may be a compiled or interpreted language.

Each such computer program can be stored on a storage medium or device (e.g., CD-ROM, hard disk or magnetic diskette) that is readable by a general or special purpose programmable computer for configuring and operating the computer when the storage medium or device is read by the computer to perform the procedures described. The system also may be implemented as a computer-readable storage medium, configured with a computer program, where the storage medium so configured causes a computer to operate in a specific and predefined manner.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the invention and that methods and structures within the scope of these claims and their equivalents be covered thereby.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for providing a leveling recommendation to a doctor for a patient's teeth, the method comprising:
inputting into a computing system an initial anterior leveling dataset of the patient's anterior teeth;
inputting into the computing system an incisor preference (b_{pref}) received from the doctor regarding a leveling position of lateral incisors;
obtaining with the computing system an average anterior leveling (μ) dataset derived from a plurality of patients;
determining a final leveling dataset based on the initial anterior leveling dataset, the incisor preference received from the doctor and the average anterior leveling dataset; and
displaying, to the doctor, a final leveling recommendation from the final leveling dataset.

2. The method of claim 1, further comprising generating an orthodontic device from the final leveling dataset.

3. The method of claim 1, wherein determining the final leveling dataset is further based on at least one latent factor.

4. The method of claim 3, wherein the at least one latent factor comprises one or more of: a country bias, a gender bias, a patient-type bias, and a doctor/gender bias.

5. The method of claim 3, wherein the at least one latent factor comprises a doctor/gender bias.

6. The method of claim 1, wherein inputting the incisor preference (b_{pref}) comprises inputting the incisor preference (b_{pref}) received from the doctor regarding the leveling position of lateral incisors with respect to a leveling position of central incisors.

7. The method of claim 1, wherein determining comprises applying matrix factorization to matrices containing the initial anterior leveling dataset, the incisor preference (b_{pref}), and the average anterior leveling dataset (μ).

8. The method of claim 1, wherein the incisor preference comprises a preference by the doctor that the lateral incisors be raised with respect to the central incisors.

9. The method of claim 1, wherein the incisor preference comprises a preference by the doctor that the lateral incisors be level with the central incisors.

10. The method of claim 1, wherein the incisor preference comprises a preference by the doctor that the lateral incisors and central incisors be leveled based gingival margins of the patient.

11. The method of claim 1, wherein the final leveling dataset comprises a matrix containing a set of possible leveling recommendations and a score for each leveling recommendation.

12. The method of claim 11, further comprising selecting the final leveling recommendation from the final leveling dataset.

13. The method of claim 12, wherein the selected final leveling recommendation comprises a highest score from the set of possible leveling recommendations.

14. The method of claim 1, wherein the final leveling recommendation is added to the average anterior leveling dataset.

15. The method of claim 1, further comprising determining a posterior leveling dataset based on the patient's posterior teeth and further wherein determining the dataset comprises determining the final leveling data set based on the initial anterior leveling dataset, the incisor preference received from the doctor, the average anterior leveling dataset, and the posterior leveling dataset.
